(19) United States
(12) Patent Application Publication
(10) Pub. No.: US 2010/0268249 A1
(43) Pub. Date: Oct. 21, 2010

(54) SURGICAL SYSTEM WITH MEDICAL MANIPULATOR AND STERILE BARRIER

(75) Inventor: J. Michael Stuart, Rio Rancho, NM (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MicroDexterity Systems, Inc., Albuquerque, NM (US)

(21) Appl. No.: 12/425,762

(22) Filed: Apr. 17, 2009

(51) Int. Cl. A61B 19/00

(52) U.S. Cl. 606/130

(57) ABSTRACT

A surgical system for use in performing medical procedures on a body of a patient is provided. The system can include a manipulator having a tool mounting arrangement including a modulated mechanical energy transmitter capable of transferring power. The manipulator is capable of moving the tool mounting arrangement with at least one degree of freedom. The system also includes a tool support including a modulated mechanical energy receiver capable of receiving power. A sterile barrier is arranged between the robotic mechanism and the tool support to isolate the robotic mechanism from the sterile environment. The tool support is engageable with the tool mounting arrangement with the sterile barrier therebetween and with the sterile barrier extending between the modulated mechanical energy transmitter and receiver. The modulated mechanical energy transmitter and the modulated mechanical energy receiver can transmit power across the sterile barrier between the manipulator and the tool support when the tool support is engaged with the tool mounting arrangement. The system can further include a retention mechanism configured for engaging the tool support with the tool mounting arrangement with the sterile barrier therebetween only when the tool support and tool mounting arrangement are in at least one desired orientation relative to each other.

### BACKGROUND OF THE INVENTION

Conventional devices which are used to perform very complex and/or physically demanding surgical procedures like neurosurgery, spine surgery, ear surgery, head and neck surgery, hand surgery and minimally invasive surgical procedures have a number of drawbacks as it relates to the dexterity of the surgeon. For example, the surgeon can easily become fatigued by the need to manually support the surgical device during its use. Additionally, the surgeon may have to orient his hands in an awkward position in order to operate the device. Furthermore, conventional devices used in such surgical procedures can produce angular magnification of errors. As a result, a surgeon has considerably less dexterity and precision when performing an operation with such surgical devices than when performing an operation by traditional techniques in which the surgeon grasps a tool directly.

Accordingly, there is an increasing interest in the use of powered manipulators, such as robotic and master-slave manipulators for supporting and manipulating surgical tools during medical procedures. Such manipulators can provide a number of advantages to both patients and medical practitioners. In particular, a master/slave controlled manipulator can enhance the dexterity of the surgeon/operator so as to allow the surgeon to manipulate a medical tool with greater dexterity than he could if he was actually holding the tool in his hands. A manipulator can also reduce the fatigue experienced by a surgeon, since it eliminates the need for the surgeon to physically support the medical tool or device during its use. Additionally, the surgeon can let go of the manipulator and perform other tasks without the medical tool undergoing movement, which increases the efficiency of the surgeon and can reduce the number of individuals that are necessary to perform a particular procedure. Thus, manipulators can allow medical procedures to be performed much more rapidly, resulting in less stress on the patient.

However, the use of powered manipulators in medical, and in particular surgical, procedures raises other issues. One such issue relates to sterilization. Medical instruments or tools that become contaminated during a medical procedure must be sterilized before being used with another patient or discarded. In most cases, discarding a powered manipulator after a single use is not economically feasible. Yet, in many cases, sterilizing a powered manipulator is also not a realistic option due to the size of the manipulator or the complexity of its electronics.

One way in which to address this issue is with a sterile barrier that isolates some of the equipment from the contaminated environment so that it does not have to be sterilized. However, it can be difficult to adapt medical manipulators so that they can operate with a sterile barrier in an efficient and cost effective manner.

### BRIEF SUMMARY OF THE INVENTION

The invention provides a surgical system for use in performing medical procedures on a body of a patient. The system includes a manipulator having a tool mounting arrangement including a power transmitter. The manipulator is capable of moving the tool mounting arrangement with at least one degree of freedom. The system has a tool support including a power receiver.

A sterile barrier is arranged between the robotic mechanism and the tool support to isolate the robotic mechanism from the sterile environment. The tool support is engageable with the tool mounting arrangement with the sterile barrier therebetween. The power transmitter and power receiver can wirelessly transmit power across the sterile barrier between the manipulator and the tool support when the tool support is engaged with the tool mounting arrangement.

The surgical system can further include a retention mechanism configured for engaging the tool support with the tool mounting arrangement with the sterile barrier therebetween only when the tool support and tool mounting arrangement are in at least one desired orientation relative to each other.

The surgical system can also be configured such that the sterile barrier has a portion thereof formed to fit tightly over either a first or second component of the retention mechanism.

### BRIEF DESCRIPTION OF THE SEVERAL VIEWS OF THE DRAWING(S)

FIG. 1 is a schematic perspective view of an exemplary surgical system with a manipulator and a sterile barrier according to the present invention.

FIG. 2 is a schematic side sectional view showing an illustrative embodiment of a tool mount and tool support with a sterile barrier arranged therebetween in which the tool mount and tool support are adapted to inductively couple across the sterile barrier and a second optical fiber connection is provided to transfer data across the sterile barrier.

FIG. 3 is a schematic side sectional view showing an illustrative embodiment of a tool mount and tool support with a sterile barrier arranged therebetween in which the tool mount and tool support are adapted to inductively couple across the sterile barrier and a second coupling between radio frequency transceivers is provided to transfer data across the sterile barrier.

FIG. 4 is a schematic side sectional view showing an illustrative embodiment of a tool mount and tool support with a sterile barrier arranged therebetween in which the tool mount and tool support are adapted to inductively couple across the sterile barrier and a second coupling between LEDs and sensor semiconductors is provided to transfer data across the sterile barrier.

FIG. 5 is a schematic side view of an alternative embodiment of a tool mount and tool support that employs a capacitive coupling across the sterile barrier.

FIG. 6 is a schematic side sectional view showing an illustrative embodiment of a tool mount and tool support with a sterile barrier arranged therebetween in which the tool mount and tool support are adapted to couple using modulated mechanical energy.

FIG. 7 is a schematic sectional plan view showing an illustrative embodiment of a retention mechanism for engaging the tool support and the tool mount.

FIG. 8 is a schematic sectional plan view showing another embodiment of a retention mechanism for engaging the tool support and the tool mount.

FIG. 9 is a schematic side sectional view of an illustrative tool mount with a sterile barrier formed over the tool mount.

FIG. 10 is a schematic side view of an illustrative tool mount and tool support in which mechanical force is transmitted across the sterile barrier in order to operate a tool.

### DETAILED DESCRIPTION OF THE INVENTION

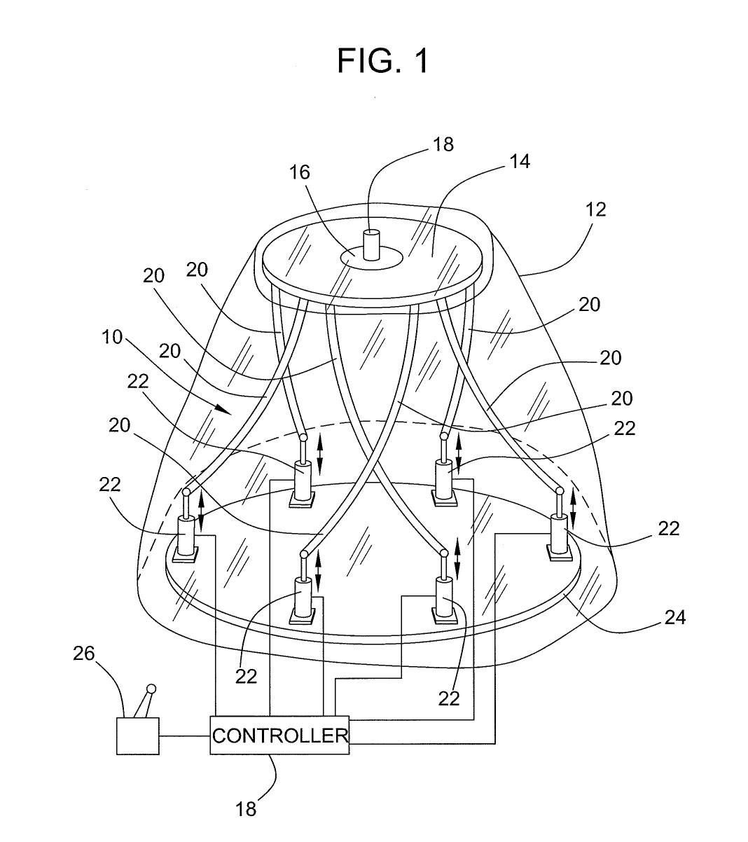

Referring now to FIG. 1 of the drawings there is shown an illustrative surgical system including a manipulator 10 that is equipped with a sterile barrier 12 in accordance with the present invention. The illustrated manipulator 10 can interchangeably support and move a medical tool with up to six degrees of freedom. While the present invention is disclosed in connection with a particular embodiment of a manipulator those skilled in the art will appreciate that is also applicable to other manipulator systems including systems which have as little as one degree of freedom. Moreover, the present invention is not limited to any particular type of medical tool. Some examples of tools that can be used include needle holders, staple or clamp appliers, probes, scissors, forceps, cautery, suction cutters, dissectors, drills, saws, lasers, ultrasonic devices and diagnostic devices.

In the illustrated embodiment, the manipulator 10 is a parallel manipulator that includes an end platform 14 that carries a tool mount 16. As described in greater detail below, the tool mount 16 mates with a tool support 18 that, in turn, carries the tool. The tool support 18 is adapted such that various different tools are attachable, detachable and re-attachable to the tool support. Alternatively, the tool and tool support could be a single integral element. The end platform 14 is supported, in this case, by six links 20. A linear actuator 22 comprising a linear motor is provided for each of the links 20. In particular, each linear actuator 22 is attached to the end of its respective link 10 that is not connected to the end platform 14. The linear actuators 22 are arranged in spaced relation from each other in a generally circular pattern about a base 24. Each link 20 can be attached to the end platform 14 using a universal joint having two degrees of rotary freedom and to its respective linear actuator 22 using a universal joint having three degrees of rotary freedom. With this arrangement, the parallel mechanism 10 can manipulate the end platform 14 with six degrees of freedom by moving the links 20 through extension and retraction of one or more of the linear actuators 22.

Depending on the desired performance, the illustrated parallel manipulator 10 can have any number of links 20 and the links can have different configurations. Moreover, the links 20 can be arranged in a variety of different geometries. Additional details regarding link geometries and the structure and operation of the illustrated parallel manipulator are provided in commonly owned U.S. Pat. No. 6,330,837, the disclosure of which is incorporated herein by reference. As noted above, the present invention is not intended to be limited to any particular type of manipulator or manipulator configuration and the parallel manipulator is being described merely to illustrate one particular implementation of the invention.

In order to provide dexterity enhancement for an operator/surgeon in performing surgical and certain interventional radiology procedures, the manipulator 10 can be used as a slave robot in a master-slave robotic system. In a master-slave robotic system, a surgeon/operator provides position input signals to the “slave” manipulator via a master or haptic interface which operates through a controller or control console. Specifically, with the manipulator 10 of the present invention serving as the slave robot, the surgeon indicates the desired movement of the tool held by the manipulator 10 through the use of an input device on a haptic interface 26 such as a six degree of freedom tool handle with or without force feedback, joystick, foot pedal or the like. The haptic interface 26 relays these signals to a controller 28, which, in turn, applies various desired predetermined adjustments to the signals prior to relaying them to the slave manipulator 10. Any haptic interface having six or more degrees of freedom (DOF) can be used to control the manipulator 10 via the controller. Examples of haptic interfaces or masters which can be used with the present invention include the Freedom 6S available from MPB Technologies of Montreal, Canada, and other haptic interfaces commercially available from Sensable Technology of Cambridge, Mass. and MicroDexterity Systems of Albuquerque, N.M.

Based on the signals provided by the controller 28, the manipulator 10 executes the desired movement or operation of the tool. Thus, any desired dexterity enhancement can be achieved by setting up the controller 28 to perform the appropriate adjustments to the signals sent from the haptic interface 26. For example, this can be accomplished by providing the controller with software which performs a desired dexterity enhancement algorithm. Software dexterity enhancement algorithms can include position scaling (typically downscaling), force scaling (up-scaling for bone and cartilage, downscaling for soft tissue), tremor filtering, gravity compensation, programmable position boundaries, motion compensation for tissue that is moving, velocity limits (e.g., preventing rapid movement into brain, nerve or spinal cord tissue after drilling through bone), and, as discussed in greater detail below, image referencing. These and other examples of possible algorithms are well known in the field of robotics and described in detail in published literature. The ZMP SynqNet® Series Motion Controllers which employ the SynqNet system and are available from Motion Engineering of Santa Barbara, Calif. are one example of a suitable controller for use with the present invention (see www.synqnet.org and www.motioneng.com). Another example of a suitable controller is the Turbo PMAC available from Delta Tau Data Systems of Northridge, Calif.

In accordance with one aspect of the present invention, the manipulator 10 can be adapted to operate with an associated sterile barrier 12 that isolates the manipulator 10 from the medical tool that is being manipulated and the patient during a medical procedure. The sterile barrier 12 protects the manipulator 10 from contamination and thus, there is no need to sterilize the manipulator after each use. The medical tools carried by the manipulator 10 which come in contact with the patient, in turn, have to be sterilized if they are to be re-used. To this end, the medical tools can be designed to be reusable, limited reuse or disposable. In the illustrated embodiment, the sterile barrier 12 is in the form of a drape that can be arranged around the manipulator 10. The sterile drape can be made of a thin, plastic material that is formed in a known manner from medical polymers.

Along with imparting motion, the manipulator 10 also can provide power to the medical tool. For instance, the medical tool can be a tool such as a saw, drill or laser that requires power to operate. Alternatively, the tool may having moving parts that are conventionally human powered (e.g., forceps, scissors, etc.), but have been adapted to be powered by an actuator. In either case, the power for operating the tools preferably is supplied through the manipulator. Additionally, it is often desirable that information or data be exchanged between the manipulator and the tool. For example, control signals may be directed from the manipulator to the tool or feedback signals generated from sensors on the tool may be directed from the tool back to the manipulator.

To allow for the transmission of power and information between the manipulator and the tool and otherwise facilitate the physical and electrical connection between the manipulator and the tool, some known surgical manipulator and sterile drape arrangements provide openings in the drape. These openings allow for a direct physical engagement between the manipulator and the tool. However, because of these openings, such drapes provide less than ideal protection against contamination. Moreover, such drape and manipulator arrangements can require more expensive tools because the tools must have electrical contacts that mate with electrical contacts on the manipulator on the other side of the sterile drape in order to transmit power between the manipulator and the tool. This expense can be a significant problem if the tools are designed to be disposable.

One significant advantage of the present invention is that the sterile barrier 12 can be designed as a continuous, solid barrier that does not have any openings. Such a solid barrier can be provided because the tool mount 16 of the manipulator 10 and the tool support 18 for the medical tool are adapted to transmit power wirelessly across a gap and through the sterile barrier 12 such that the sterile barrier can extend unbroken between the tool mount 16 and the tool support 18. In this regard, the tool mount 16 includes a power transmitter and the tool support 18 includes a power receiver.

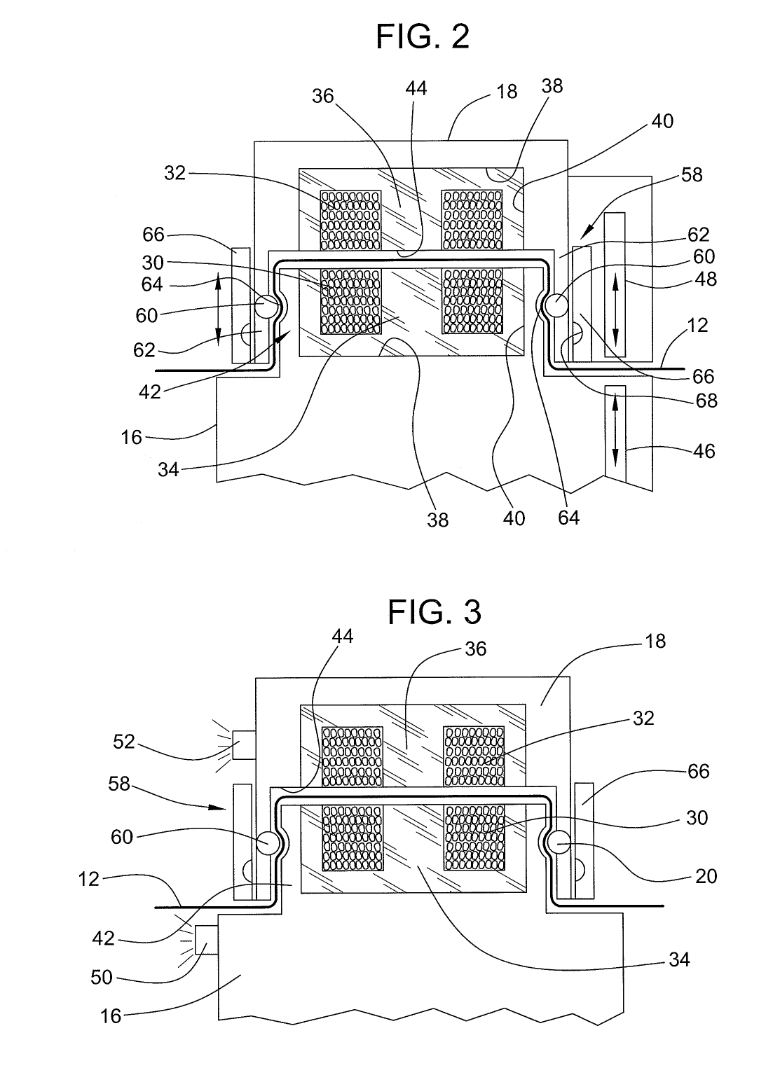

In the illustrated embodiment, this wireless and contactless transmission of power is achieved via inductive coupling between the tool mount 16 and the tool support 18. As shown in FIG. 2, the inductive coupling includes a primary or first coil or winding 30 that is carried by the tool mount 16 of the manipulator and a secondary or second coil or winding 32 that is carried by the tool support 18. In this case, the primary and secondary coils 30, 32 are each wound around a respective central cylindrical rod 34, 36 that is made of magnetic material. The rods 34, 26 for the primary and secondary coils 30, 32 are arranged in respective cups each of which has a circular back wall 38 from which the corresponding rod extends and a cylindrical sidewall 40 having a height equal to that of the corresponding rod. Both cups are open at one end and are made of magnetic material. The primary coil, rod and core 30, 34, 38, 40 on the tool mount 16 and the secondary coil, rod and core 32, 36, 38, 40 on the tool support 18 are, in this case, substantially identical in construction.

As shown in FIG. 2, the primary coil, rod and core 30, 34, 38, 40 are arranged at a mounting end of tool mount 16 of the manipulator 10 with the open end of the core facing outward. Similarly, the secondary coil, rod and core 32, 36, 38, 40 are arranged at a mounting end of the tool support 18 with the open end of the secondary coil core also facing outward. In the illustrated embodiment, the tool mount 16 of the manipulator comprises a mounting pin 42 and the tool support 18 includes a mating receptacle 44 for receiving the mounting pin. Thus, in this case, the primary coil, rod and core 30, 34, 38, 40 are arranged on the distal end of the mounting pin 42 and the secondary coil, rod and core 32, 36, 38, 40 are arranged in the base of the receptacle 44.

The tool support receptacle and the mounting pin are configured such that the mounting pin can engage in the receptacle with the sterile barrier draped over the mounting pin as shown in FIG. 2. Thus, when the mounting pin 42 is engaged in the receptacle 44, the sterile barrier 12 extends between and separates the mounting pin from the receptacle so that there is no direct physical contact between the tool mount 16 and the tool support 18. In the engaged position of the mounting pin and receptacle, the primary coil, rod and core 30, 34, 38, 40 and the secondary coil, rod and core 32, 36, 38, 40 face each other and are coaxially aligned. In this position, the primary and secondary coil, rod and core are adjacent each other, but with a small gap between them through which the sterile barrier 12 extends. It is preferred that the gap be approximately 0.05 inches or less. As will be appreciated by those skilled in the art, the primary and secondary coils, rods and cores do not have to be identical (although it is helpful if they are similar) nor do they have to align perfectly.

When the mounting pin 42 is engaged with the receptacle 44 and in the position shown in FIGS. 2 and 3, electric power can be transmitted inductively between the primary and secondary “couplings” (i.e., the coils, rods and cores) through the sterile barrier 12. For example, AC power in the range of 50 Hz-100 kHz can be transmitted from the manipulator to the tool. Magnetic leakage can be kept to a minimum through good core alignment and keeping the gap between the primary and secondary coils, rods and cores relatively small. When using an inductive coupling to provide the power transmission between the tool mount and the tool support it is preferred that the sterile barrier be made of a material that has a low dissipation factor in the radio frequency region of the electromagnetic spectrum.

In the illustrated embodiment, a second wireless and contactless “coupling” between the tool support 16 and the tool mount 18 is used to transmit information or data between the two components. Such information or data could comprise control signals, feedback information, etc. such as for use with “smart” medical instruments. In the embodiment shown in FIG. 2, the data transfer is achieved using optical fibers. In particular, the tool mount 16 carries a first optical fiber 46 that aligns with a second optical fiber 48 carried on the tool support 18 when the tool support and tool mount are engaged with a small gap being provided between the ends of the optical fibers through which the sterile barrier 12 can extend. The data moves between the tool support 18 and the tool mount 16 via modulated light that is transmitted through the sterile barrier 12 from one optical fiber 46, 48 to the other. Of course, the data transmission can be in either direction, i.e. from the tool support to the tool mount or from the tool mount to the tool support. When light transmitted across the sterile barrier is used to for data transmission, the sterile barrier is preferably made of a material that has a high transmissibility for light. Of course, the sterile barrier can be formulated to meet the particular needs of the couplings used to transmit power and data across the sterile barrier. For instance, if an electromagnetic coupling was used for transmission of power but something other than a fiber optic coupling was used for data transmission, a high transmissibility of light may not be a necessary property if a fiber optic coupling is not used for data transmission but a low dissipation factor in the radio frequency region of the electromagnetic spectrum would be a desirable property.

Other wireless and contactless transmission methods also could be used for the data “coupling” in place of or in combination with the optical fiber coupling. A radio frequency (“RF”) coupling also could be used. In particular, as shown in the embodiment of FIG. 3, the tool mount 16 could carry a first RF transceiver 50 and the tool support 18 could carry a second RF transceiver 52. The first and second transceivers 50, 52 are configured such that they can communicate when the tool support 16 is engaged with the tool mount 18 and power is supplied to the tool support. When they are in communication, the RF transceivers 50, 52 can be used to transmit data signals between the two components. The RF transceivers 50, 52 could be arranged in other locations as well so long as one is provided on each side of the sterile barrier 12 and they are able to communicate when the tool support 16 is engaged with the tool mount 18.

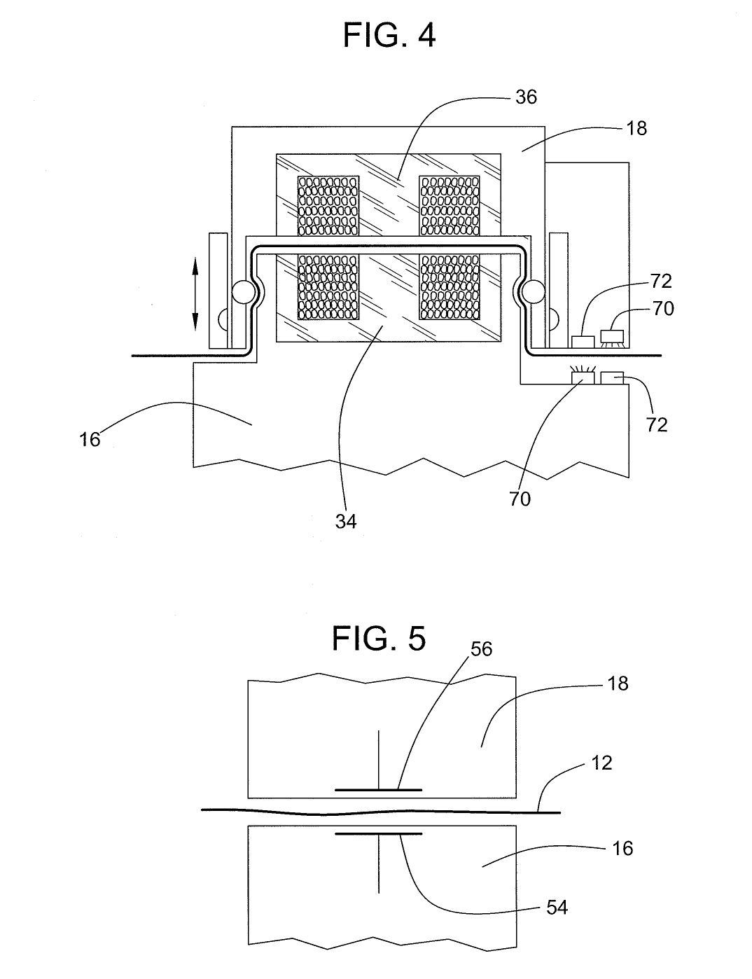

Another way in which light could be used to transmit data across the sterile barrier 12 is by using LEDs 70 and sensor semiconductors 72. For instance, both the tool support 18 and tool mount 16 could be provided with LEDs 70 and sensor semiconductors 72 that would be in alignment (the LEDs 70 of one component aligned with the sensor semiconductors 72 of the other component) when the tool mount 16 and tool support 18 are engaged to allow for data transfer in both directions across the sterile barrier 12. Such an arrangement is schematically shown in FIG. 4. Alternatively, the data transfer could be accomplished, like the power transfer, by inductive coupling. In such a case, the data transfer could be via electrical signals at a different frequency band than the power coupling in the form of electrical signal using broadband, CDMA, UWB or some other high signal to noise protocol.

As an alternative to inductive coupling, capacitive coupling could be used to transmit the power and/or data between the tool mount and the tool support. In particular, as shown in the embodiment of FIG. 5, the tool mount 16 could carry a first capacitor plate 54 and the tool support 18 could carry a second capacitor plate 56. When the tool support 18 and tool mount 16 are engaged, the first and second capacitor plates 54, 56 would be arranged in close proximity with the sterile barrier 12 extending therebetween and acting as a dielectric material so as to facilitate electrical power transfer and/or data transfer between the two components.

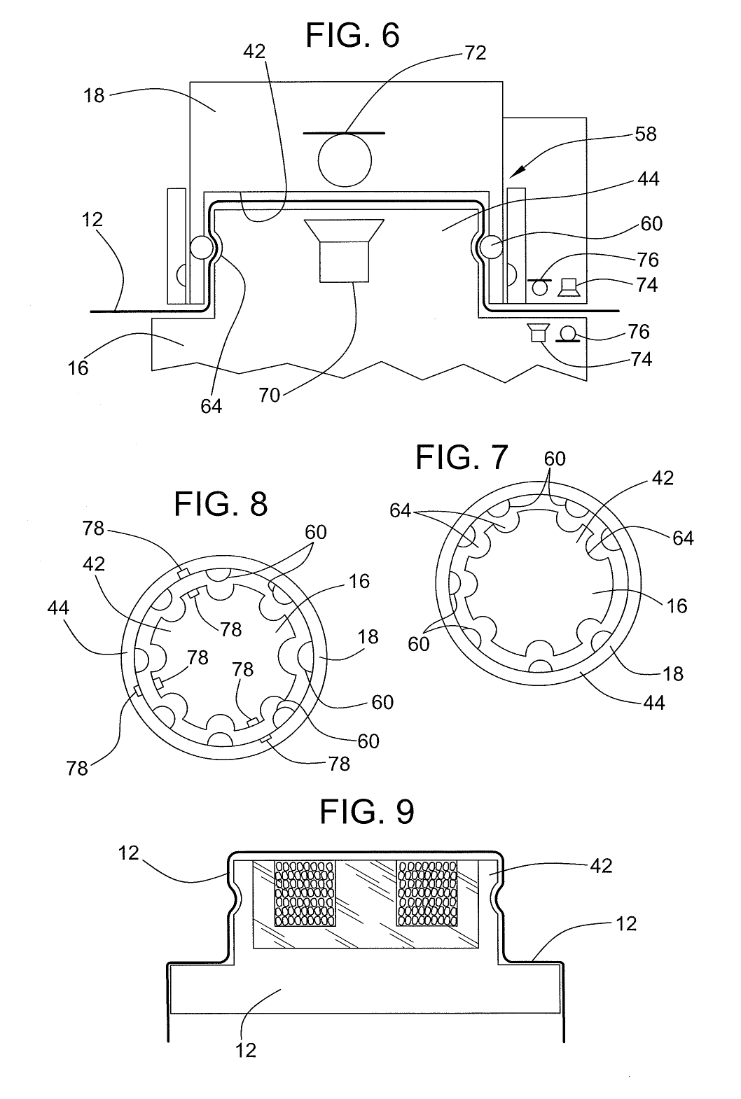

Another alternative for the couplings for the power and/or data transfer between the tool mount 16 and the tool support 18 across the sterile barrier 12 is the use of a coupling based on ultrasound or other forms of modulated mechanical energy. For example, as shown in the embodiment of FIG. 6, to permit the power to be transferred from the tool mount 16 to the tool support 18, the tool mount 16 can be equipped with an ultrasonic transmitter 70 and the tool support can be equipped with an ultrasonic receiver 72. The illustrated embodiment includes a separate “channel” for data transfer with one a second of modulated mechanical energy transmitters and receivers for data transfer across the sterile barrier. If the data transfer is to be in both directions, such as with a data coupling where data is transferred both to and from the tool support, both the tool mount 16 and the tool support 18 could be provided with ultrasonic data transmitters 74 and receivers 76 such as shown in FIG. 6.

The ultrasonic transmitters and receivers 70, 72, 74, 76 should be arranged on the components such that when the tool mount 16 and the tool support 18 are engaged with the sterile barrier 12 therebetween, the ultrasonic signals produced by the transmitters 70, 74 are received by the corresponding receivers 72, 76 across the sterile barrier 12 and the gap between the components necessary to accommodate the sterile barrier 12. Other forms of modulated mechanical energy could also be used to provide the necessary couplings across the barrier. For example, modulated pressure transmitted through the barrier could be used to provide the data coupling between the tool support and tool mount. Alternatively, power and data could be transferred using the same modulated mechanical energy transmitters and receivers or an alternative type of wireless transfer could be used for one of the channels such as an inductive, capacitive, RF or modulated light.

For securing the tool mount 16 to the tool support 18, a retention mechanism 58 can be provided which permits the tool support to be attached to the tool mount while maintaining the arrangement of the sterile barrier 12 between the two components. In the illustrated embodiment, as shown in FIGS. 2 and 3, the tool support receptacle 44 includes a retention mechanism 58 that engages the mounting pin 42 of the tool mount 16 while maintaining the desired gap between the primary and secondary primary and secondary coils, rods and cores. The retention mechanism 58 includes a plurality of locking balls 60 that are carried by one of the tool mount or tool support 16, 18 and are captured in openings in the other of the two components. In this instance, the retention mechanism 58 includes a plurality of locking balls 60 arranged in an annular pattern around the sidewall 62 of the receptacle 44. Each locking ball 60 is received in a respective opening 64 in the sidewall of the mounting pin 42 of the tool mount 16 and is movable into and out of that opening in a radial direction relative to the sidewall of the receptacle between locked and unlocked positions. Alternatively, the locking balls 60 could engage in an annular round-bottomed groove in the outer surface of the sidewall of the mounting pin.

In the locked position, the locking balls 60 engage in the respective openings 64 in the mounting pin 42 so as to prevent movement of the tool support 18 relative to the tool mount 16. According to one embodiment, eight locking balls 60 are spaced around the receptacle 44 each of which engages a respective opening in the mounting pin 42. Providing eight points of engagement provides a highly precise engagement in that tool support 18 is locked to the tool mount 16 at eight separate positions about the rotary degree of freedom.

In the illustrated embodiment, the locking balls 60 are held in the locked position by an annular retention sleeve 66 that bears against the locking balls 60 and pushes them radially inward into engagement with the corresponding openings 64 on the mounting pin 42. This retention sleeve 66 is supported in surrounding relation on the tool support receptacle 44 for longitudinal movement relative to the sidewall of the receptacle. In this case, to unlock the locking balls 60, the retention sleeve 66 is pulled back in a direction away from the mounting pin 42 until a groove 68 on the inside surface of the sleeve is aligned with the locking balls. When the groove 68 on the inside surface of the sleeve 66 aligns with the locking balls 60, the locking balls are able to move radially outward into their unlocked position in which the balls are engaged with the groove on the latch and out of engagement with the openings 64 on the mounting pin. The mounting pin 42 can then be pulled out of the receptacle 44. To lock the mounting pin 42 in the receptacle 44, the retention sleeve 66 is slid forward on the receptacle 44 so that the groove 68 on the sleeve moves out of alignment with the locking balls 60 and the inside surface of the retention sleeve cams or pushes the locking balls radially inward into engagement with the openings 64 on the mounting pin. The locking balls 60 are pushed radially outward by the mounting pin 42 as it is inserted into and pulled out of the receptacle 44. The locking balls 60 should be free to move radially outward when the retention mechanism 58 is unlocked and to move radially inward when the retention mechanism is unlocked. The retention sleeve 66 is preferably spring loaded towards its locked position.

Of course, other types of retention mechanisms could be used and those skilled in the art will appreciate that the present invention is not necessarily limited to any particular type of retention mechanism. For example, if the locking balls 60 are carried on the tool mount mounting pin 42 rather than the tool support receptacle 44 a cam device comprising a secondary pin portion in the mounting pin could be used to move the locking balls into engagement with complementary openings in the tool support receptacle. Other retention mechanisms could be used as well. Additionally, it will be appreciated that the retention mechanism could be manually operable or automatically operable via electric or some other type of actuators.

Particularly if a multi-point retention mechanism is used, the cylindrical sidewall of the tool support receptacle 44 can be replaced by a plurality of spaced post elements each of which extends parallel to the center rod element 36. For example, if a retention mechanism 58 having eight locking balls is used, the tool support receptacle could be defined by eight spaced apart posts with each post carrying one of the locking balls. With such an arrangement, the magnetic circuit created by the inductive coupling would be defined in eight positions.

In order to ensure that tool mount 16 and tool support 18 can engage in only a single position or in a small number of positions relative to each other, the locking balls 60 and mating openings 64 in which the locking balls are received can be arranged so as to provide a “keyed” type of engagement between the tool mount and tool support. In particular, as shown schematically in FIG. 7, the locking balls 60 and mating openings 64 can be arranged in an irregular pattern around the circumference of the retention mechanism 58. As will be appreciated, if eight locking balls 60 and openings 64 are provided and the locking balls and openings are spaced in a regular pattern equidistant from each other the mounting pin 42 and tool support receptacle 44 could mount in at least eight different angular positions relative to each other. However, the use of an irregular pattern for the locking balls 60 and openings 64 can significantly limit the number of positions in which the mounting pin 42 and tool support receptacle 44 can engage. Such a limitation can be important because the medical tool carried by the tool support 18 may need to be in a specific orientation relative to the manipulator 10.

The specific irregular pattern shown in FIG. 7 permits the balls and openings to be aligned only when the mounting pin 16 and the tool support receptacle 18 are in a single angular position relative to each other. Of course, this could be accomplished other ways. For instance, one of the locking balls 60 and one of the openings 64 for receiving the locking balls could be a different size than the rest such as shown in FIG. 8. Such an arrangement would also only allow the mounting pin 42 and tool support receptacle 44 to engage in a single angular position relative to each other. Moreover, the locking balls 60 and openings 64 could be positioned or sized so that the mounting pin 42 and tool support receptacle 44 could engage in more than one, but still in a relatively small number of positions.

Alternatively or additionally, sensors 78 that are capable of operating across the sterile barrier 12 could be used to sense the position of the tool mount 16 relative to the tool support 18 (or even the tool carried by the tool support). For example, the mounting pin 42 and tool support receptacle 44 could incorporate sensors 78 (shown schematically in FIG. 8) that would read the angular position of the two components relative to each other when the components are engaged. If the retention mechanism 58 enables the mounting pin 42 and tool support receptacle 44 to only engage in certain specific orientations the sensors 78 could determine in which of those orientations the components are engaged. The data regarding the relative orientation of the two components could then be communicated to the controller 28 associated with the manipulator 10 so that it could be taken into account when the controller is directing movement of the tool carried by the manipulator. The data regarding the relative orientation of the mounting pin 42 and tool support receptacle 44 could also be used by the controller 28 to determine if the two components were properly engaged by comparing the sensed orientation with the known one or more proper orientations for engagement of the two components. The sensors could be based on fiber optics, LEDs or any other suitable technology that could operate across the sterile barrier.

To help facilitate engagement of the tool support 16 with the tool mount 18 as well as full range of movement of the manipulator 10, the sterile barrier 12 could be formed so as to fit tightly over the tool mount 18 on the manipulator 10. More specifically, at least a portion of the sterile barrier 12 could be formed thermally or via some other method to fit tightly over the mounting pin as shown in FIG. 9. Such a configuration of the sterile barrier 12 will help define the specific location on the sterile barrier that should be placed over the mounting pin 42 in order to provide optimal movement of the manipulator 10. Forming the sterile barrier 12 to fit over the mounting pin 42 will also help ensure that it does not become wrinkled in the space between the tool mount 16 and the tool support 18 as wrinkles could degrade the ability of the power and data couplings to operate across the sterile barrier 12.

In order to sense the forces being applied at the medical tool, it is preferred that the system be adapted to sense force across the sterile barrier 12. One way in which this can be accomplished is to provide a force sensor on the non-sterile side of the system on the manipulator 10 that is arranged and configured to sense force being applied at the tool. With such an arrangement, the sterile barrier 12 should be sufficiently flexible that it provides a negligible force component to the overall force being sensed by the force sensor inside the sterile barrier. Alternatively, the force sensor could be incorporated into the tool on the sterile side of the sterile barrier 12 with the force data being transmitted back to the manipulator 10 through the sterile barrier 12 via the data “coupling” across the sterile barrier.

Often the tool operated by the manipulator 10 is also independently movable. Examples of such tools are scissors, forceps and the like. In order to permit the manipulator 10 to drive this independent movement (i.e., operate the scissors), the system could be designed to transmit mechanical movement through the sterile barrier 12. In particular, the sterile barrier 12 could be made sufficiently flexible and the tool mount 16 and tool support 18 could be configured such that moving components on the tool mount 16 would deflect the flexible barrier in such a manner as to actuate the tool. The movement of the moving components on the tool mount 16 would be directed by the manipulator controller 28 so that actuation of the tool would be automatically directed by the manipulator 10 and the controller 28.

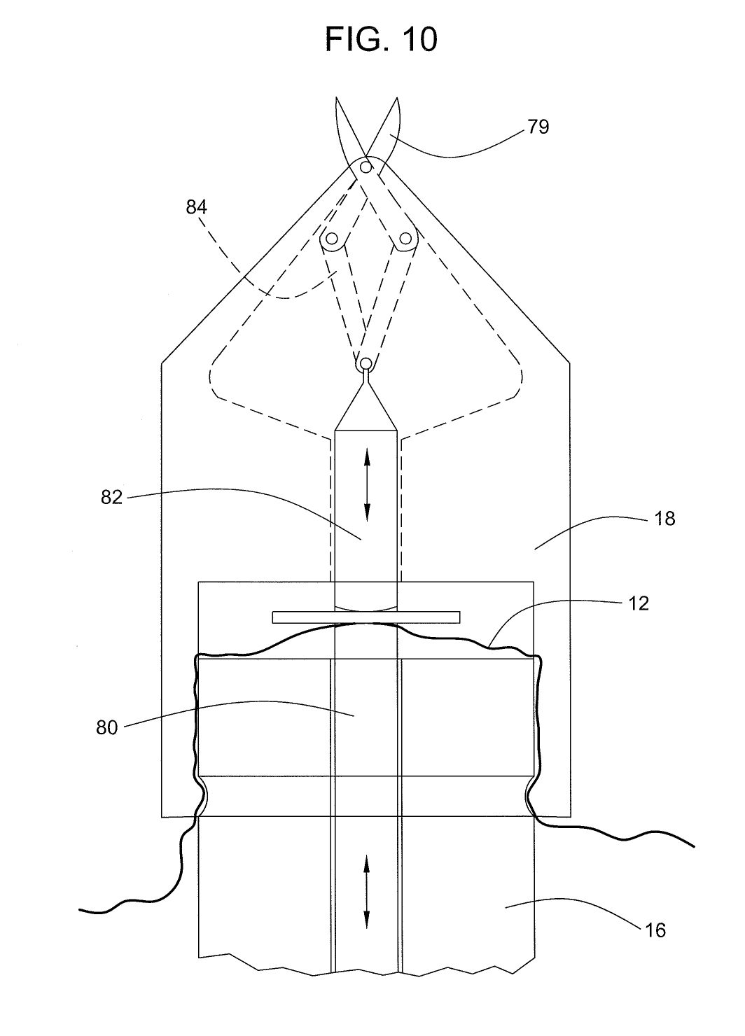

An exemplary embodiment of an arrangement of the tool mount 16 and tool support 18 that would allow for mechanical actuation of a tool such as scissors 79 across the sterile barrier 12 is shown in FIG. 10. In the illustrated embodiment, the tool mount 16 carries a longitudinally movable piston 80 that moves between extended and retracted positions as directed by the surgeon and/or manipulator controller. When the tool support 18 is engaged with the tool mount 16, an engagement end of the piston 80 aligns with an engagement end of a longitudinally movable plunger 82 carried by the tool support 18 with the sterile barrier 12 extending between the engagement ends of the piston 80 and the plunger 82. The end of the plunger 82 opposite the engagement end is connected to a toggle linkage 84 that drives the opening and closing of the scissors 79.

As noted above, the sterile barrier 12 is flexible such that when the piston 80 carried by the tool mount 16 extends the sterile barrier 12 deflects allowing the piston 80 to push on the plunger 82. The pushing action that is transmitted across the sterile barrier 12 drives the plunger 82 forward. This, in turn, operates the toggle linkage 84 so as to close the scissor mechanism. Rearward movement of the plunger 82 operates the toggle linkage 84 to open the scissor mechanism. This rearward movement could be generated, for example, by a spring that normally biases the plunger 82 rearward so as to keep the scissors 79 open. The spring force should be such that it can be overcome by the force applied by the piston 80 when it extends and pushes on the plunger 82 to close the scissor mechanism via the toggle linkage 84. When the force driving the piston 80 is relieved or removed, the plunger 82 moves rearward under the force of the spring driving the opening of the scissor mechanism. The spring then holds the scissors 79 open until the surgeon and/or manipulator controller again directs closing of the scissors and drives the piston 80 forward. The mechanism shown in FIG. 10 could be used to drive other “open and close” type tools such as forceps or other grasping tools.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A surgical system for use in performing medical procedures on a body of a patient, the system comprising:
a manipulator having a tool mounting arrangement including a modulated mechanical energy transmitter capable of transferring power, the manipulator being capable of moving the tool mounting arrangement with at least one degree of freedom;
a tool support including a modulated mechanical energy receiver capable of receiving power; and
a sterile barrier arranged between the robotic mechanism and the tool support to isolate the robotic mechanism from the sterile environment;
the tool support being engageable with the tool mounting arrangement with the sterile barrier therebetween and with the sterile barrier extending between the modulated mechanical energy transmitter and receiver; and
wherein the modulated mechanical energy transmitter and the modulated mechanical energy receiver can transmit power across the sterile barrier between the manipulator and the tool support when the tool support is engaged with the tool mounting arrangement.

2. The system of claim 1, wherein the modulated mechanical energy transmitter and receiver can wirelessly transmit data across the sterile barrier between the manipulator and the tool support when the tool support is engaged with the manipulator.

3. The system of claim 1, wherein the manipulator and tool support further include a data channel comprising a first data transmitter carried by the tool support and a second data receiver carried by the manipulator and wherein data is transferable from the tool support to the manipulator across the sterile barrier when the tool support is engaged with the tool mounting arrangement.

4. The system of claim 1, further comprising a retention mechanism including a first component carried by the tool mounting arrangement and a second component carried by the tool support, the retention mechanism being configured for engaging the tool support with the tool mounting arrangement with the sterile barrier therebetween only when the tool support and tool mounting arrangement are in at least one desired orientation relative to each other.

5. The system of claim 1, further comprising a retention mechanism including a first component carried by the tool mounting arrangement and a second component carried by the tool support, the retention mechanism being configured for engaging the tool support with the tool mounting arrangement with the sterile barrier therebetween, the sterile barrier having a portion thereof formed to fit tightly over one of the first and second components of the retention mechanism

6. The system of claim 1, further comprising a medical tool supported on the tool support.

7. The system of claim 6, wherein the tool is detachably and reattachably supported on the tool support.

8. The system of claim 6, wherein the tool is independently movable upon actuation and the tool mounting arrangement includes moving components for deflecting the sterile barrier and engaging the tool in such a manner so to actuate movement of the tool.

9. The system of claim 5, further comprising sensors carried by the tool mounting arrangement and the tool support for sensing the orientation of the tool mount and the tool support relative to each other.

10. The system of claim 1, wherein the modulated mechanical energy comprises ultrasonic signals.

11. A surgical system for use in performing medical procedures on a body of a patient, the system comprising:
a manipulator having a tool mounting arrangement including a power transmitter, the manipulator being capable of moving the tool mounting arrangement with at least one degree of freedom;
a tool support including a power receiver;
a sterile barrier arranged between the robotic mechanism and the tool support to isolate the robotic mechanism from the sterile environment; and
a retention mechanism including a first component carried by the tool mounting arrangement and a second component carried by the tool support, the retention mechanism being configured for engaging the tool support with the tool mounting arrangement with the sterile barrier therebetween only when the tool support and tool mounting arrangement are in at least one desired orientation relative to each other; and
wherein the power transmitter and power receiver can wirelessly transmit power across the sterile barrier between the manipulator and the tool support when the tool support is engaged with the tool mounting arrangement.

12. The system of claim 11, further comprising sensors carried by the tool mounting arrangement and the tool support for sensing the orientation of the tool mounting arrangement and the tool support relative to each other.

13. The system of claim 11, wherein the retention mechanism includes a plurality of locking balls carried by one of the first and second components that are receivable in a plurality of openings in the other of the first and second components.

14. The system of claim 13, wherein the locking balls and openings are arranged in an irregular pattern such that the tool support and the tool mounting arrangement are only when the tool support and tool mounting arrangement are in at least one desired orientation relative to each other.

15. The system of claim 13, wherein at least one of the locking balls and at least one of the openings is a relatively larger than the rest of the locking balls and openings such that the tool support and the tool mounting arrangement are only when the tool support and tool mounting arrangement are in at least one desired orientation relative to each other.

16. The system of claim 11, wherein one of the first and second components comprises a mounting pin and the other of the first and second components comprises a receptacle.

17. The system of claim 11, wherein the retention mechanism is configured for engaging the tool support with the tool mounting arrangement with the sterile barrier therebetween only when the tool support and tool mounting arrangement are in a discrete plurality of orientations relative to each other.

18. The system of claim 11, wherein a portion of the sterile barrier is formed to fit tightly over one of the first and second components of the retention mechanism.

19. A surgical system for use in performing medical procedures on a body of a patient, the system comprising:
a manipulator having a tool mounting arrangement including a power transmitter, the manipulator being capable of moving the tool mounting arrangement with at least one degree of freedom;
a tool support including a power receiver;
a sterile barrier arranged between the robotic mechanism and the tool support to isolate the robotic mechanism from the sterile environment;
a retention mechanism including a first component carried by the tool mounting arrangement and a second component carried by the tool support, the retention mechanism being configured for engaging the tool support with the tool mounting arrangement with the sterile barrier therebetween, the sterile barrier having a portion thereof formed to fit tightly over one of the first and second components of the retention mechanism; and
wherein the power transmitter and power receiver can wirelessly transmit power across the sterile barrier between the manipulator and the tool support when the tool support is engaged with the tool mounting arrangement.

20. The system of claim 19, wherein the manipulator and tool support further include a data channel comprising a first data transmitter carried by the tool support and a second data receiver carried by the manipulator and wherein data is transferable from the tool support to the manipulator across the sterile barrier when the tool support is engaged with the tool mounting arrangement.

21. The system of claim 19, further comprising a medical tool supported on the tool support.

22. The system of claim 21, wherein the tool is detachably and reattachably supported on the tool support.

23. The system of claim 21, wherein the tool is independently movable upon actuation and the tool mount includes moving components for deflecting the sterile barrier and engaging the tool in such a manner so to actuate movement of the tool.

24. The system of claim 19, wherein one of the first and second components comprises a mounting pin and the other of the first and second components comprises a receptacle.

25. A method for transmitting power between a manipulator in a non-sterile environment and a tool support in a sterile environment, the method comprising:
arranging a sterile barrier between the manipulator and the tool support;
engaging the tool mount with a mounting arrangement on the manipulator with the sterile barrier extending between the engaged tool mount and the manipulator; and
transmitting power via modulated mechanical energy from a modulated mechanical energy transmitter carried by the mounting arrangement on the manipulator to a modulated mechanical energy receiver carried by the tool mount such that power can be transmitted between the manipulator and the tool support without disrupting the physical integrity of the sterile barrier disposed therebetween.
